(19) United States
(12) Patent Application Publication
(10) Pub. No.: US 2004/0077939 A1
(43) Pub. Date: Apr. 22, 2004

(54) Device and method for controlling surgical instruments

(76) Inventor: Rainer Graumann, Hochstadt (DE)

Correspondence Address: SCHIFF HARDIN, LLP, PATENT DEPARTMENT, 6600 SEARS TOWER, CHICAGO, IL, 60606-6473, US

(21) Appl. No.: 10/468,869

(22) PCT Filed: Feb. 20, 2002
(86) PCT No.: PCT/DE02/00615

(30) Foreign Application Priority Data
Feb. 22, 2001 (DE) 10108547.8

(51) Int. Cl. A61B 5/05; A61B 19/00

(52) U.S. Cl. 600/424; 606/130

(57) ABSTRACT

The invention relates to an operation system for performing surgical interventions by using a surgical instrument (3) on a patient (1). The operation system comprises an X-ray unit (2) for taking an X-ray image of at least one area of the patient (1), whereby the recorded X-ray image is pictorially represented by an output device (11). The operation system also comprises a position detection system for detecting the position of the X-ray unit (2) as well as the position of the surgical instrument (3), and comprises a processing device (10) for processing the detected positions and for continuosly superimposing a representation of the surgical instrument (3) in the displayed the X-ray image. The superimposition of the X-ray image and of the representation of surgical instrument ensues to-scale according to the actual spatial positions between the position of the recorded area of the patient (1) and of the position of the surgical instrument (3). The invention also relates to a method, which is used in the operation system and which is provided for controlling a surgical instrument during the intervention.

The present invention concerns an operation system for implementing operational procedures on a patient and a method used in this operation system for controlling a surgical instrument during an operational procedure.

In an operational procedure on a patient, the control of surgical instruments (such as, for example, laproscopes, endoscopes, needles, robots, and so on) ensues either on the basis of direct sight such as, for example, with a laproscope and endoscope, pre-operative imaging such as, for example, robots in orthopedics, or intra-operative imaging, for example, during the needle biopsy in computer tomography monitoring (CT) and ultrasound monitoring.

Operations on the knee (in what is called Total Knee Replacement) and on the hip (in what is called Total Hip Replacement) are cited as examples of a robot-aided operational procedure. Such procedures are implemented in the prior art exclusively using pre-operative CT x-ray images, i.e. the recording of the area to be operated on ensues prior to the operational procedure. However, the disadvantage of this method is that it is associated with an elaborate registration procedure.

However, other, more complex procedures require running monitoring images during the procedure in order to ensure a safe implementation. This is for example the case in vertebroplasty, in which a sterile substance (cement) is filled into a vertebral body that has become brittle, in order to stabilize this vertebral body.

The object of the present invention is to provide an operation system for implementation of operational procedures on a patient, and a method used in this operation system for controlling surgical instruments during the procedure, in which a reliable and exact control of the surgical instruments is enabled during the operational procedure on the basis of intra-operative x-ray images.

This object is achieved via an operation system for the implementation of operational procedures on a patient by means of a surgical instrument according to the attached claim 1, and via a method used in this operation system for controlling a surgical instrument during an operational procedure according to the attached claim 5.

The inventive operation system for implementation of operational procedures on a patient by means of a surgical instrument comprises an x-ray device to acquire an x-ray image of at least one area (body part, organ) of the patient (whereby the acquired x-ray image is graphically displayed via an output device), and a position detection system to detect the position of the x-ray device during the recording, and to continuously detect the position of the surgical instrument during the operational procedure.

The detected positions are processed by a processing device; the processing device thereby continually mixes an image of the surgical instrument into the displayed x-ray image during the operational procedure. The mixing of the x-ray image and the display of the surgical instrument thereby ensues in the correct positional arrangement of the actual spatial positions between the position of the recorded area of the patient and the position of the surgical instrument.

According to the present invention, an x-ray recording is made of the patient or, respectively, of the area (body part, for example organ) in which the surgical procedure should ensue, and the spatial position from which the x-ray recording was made is determined by means of the position detection system. During the surgical procedure, the current position of at least one surgical instrument is determined by the position detection system, and a corresponding image (meaning the image of the surgical instrument) is mixed into the display of the x-ray recording. The mixing thereby ensues in the correct positional arrangement in the relative position, in that the surgical instrument is actually in the appertaining area, meaning that the indicated spatial relationship between the surgical instrument and the body section acquired by the x-ray image is the same as the actual spatial relationship.

The surgical instrument is directed by a robot or manipulator, such that, in part, determined procedures can be implemented automatically or, respectively, with a substantially higher precision than in a manual procedure.

A robot enables the automatic implementation of determined procedures, for example the milling of determined areas of the hip in a hip operation, where precisely defined areas are milled.

In contrast, a manipulator comprises a robot arm that is directed, for example, by the surgeon via a joystick. A manipulator enables the surgeon to operate essentially as precisely as by hand, since, for example, the instrument can be precisely directed by the manipulator due to a translation, and tremors of the surgeon can be compensated if necessary.

The advantage of the present invention is that, during the surgical procedure, current x-ray images can be made for monitoring in order to track the progress of the operational procedure. The progression of the operation (that is thus visible on the x-ray images) can thereby be comprised in the direction of the surgical instrument.

A further advantage of the present invention is that some of the procedures on the skeleton previously implemented manually can be implemented with the aid of robots, since a reliable and adequate imaging during the operation is available according to the present invention during the operation (i.e. intra-operative) via x-ray images, for example three-dimensional x-ray images.

A use of robots or, respectively, manipulators is thus enabled in operational procedures that require a continuous monitoring via x-ray images.

Advantageous embodiments of the present invention are reproduced in the respective subclaims.

The position detection system comprises comprises [sic] a first position marker that is applied to the x-ray device (for example a C-arm x-ray device), a second position marker that is applied to the surgical instrument or to the robot/manipulator that directs the instrument, and a position detection device to detect the respective positions of the x-ray device and the surgical instrument. The spatial relation between the first position marker and the spatial position of the body part of the patient that is recorded by the x-ray device can be known, and the spatial relationship between the second position marker and the operating area (for example, the tip of the surgical instrument) can be known.

For example, the position detection system can be based on an optical, an acoustic, or an electromagnetic system.

To incorporate, for example, patient motion during the operation, the patient or, respectively, the area of the patient that is detected in terms of x-ray images, is likewise detected by means of the position detection system. In addition, a third position marker to detect the patient position (or, respectively, the position of the appropriate body part) is applied in the vicinity of the corresponding area, whereby the position of the third position sensor is continually detected, and the processing device takes into account as needed the patient motion in the displayed image. The third position marker must thereby be fixed in relation to the corresponding area (for example, knee); the spatial relationship between the third position sensor and the corresponding body part is likewise hereby known.

Given utilization of a robot, the inventive operation system comprises a control device to automatically control the robot in an automatic operational procedure (for example, specified above). The control device thereby monitors the recorded x-ray data set (for example, the x-ray image), the position of the area that is shown with the x-ray image, and the position of the surgical instrument.

Given utilization of a manipulator, the operational procedure can be automatically monitored by the control device, such that, for example, the manipulator can only address a restricted or predetermined operating area, in order to avoid unintended injury to the patient.

The present invention is subsequently more closely explained using a preferred exemplary embodiment, with reference to the attached drawings, in which are shown:

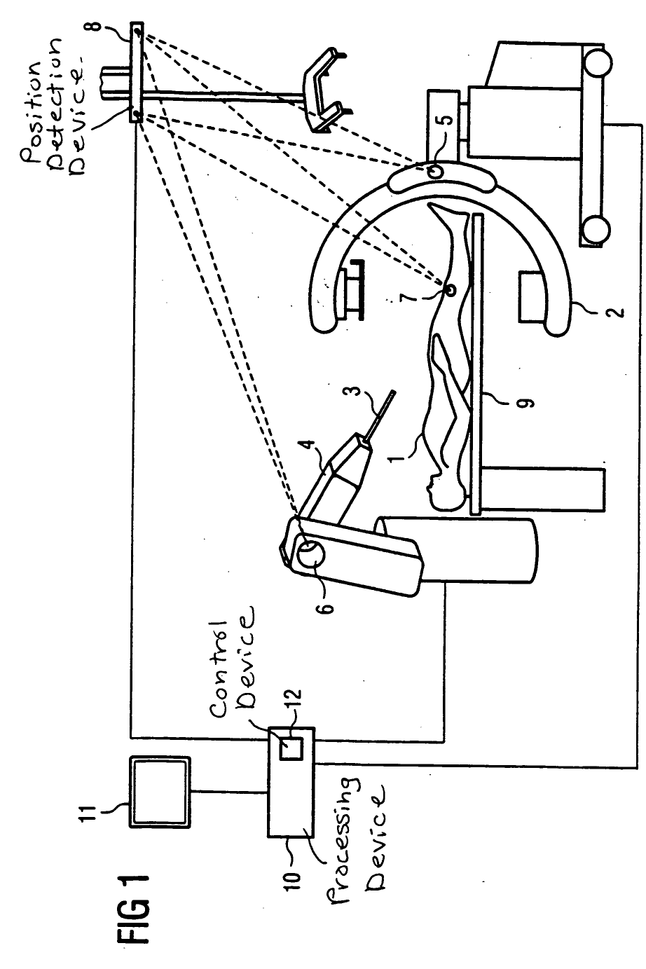

FIG. 1 a schematic representation of the operation system according to the present invention, and

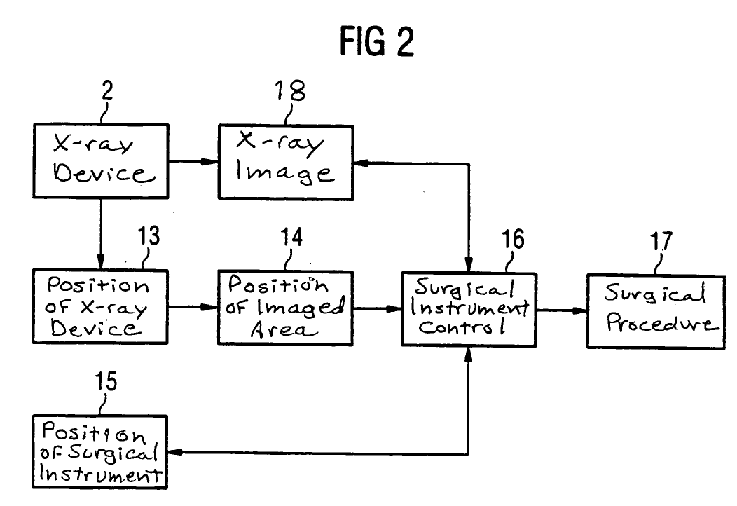

FIG. 2 a function diagram that shows the concept of the present invention.

The inventive operation system is subsequently specified using FIG. 1.

The inventive operation system comprises an x-ray device 2, in the example a mobile c-arm x-ray device for recording two- and three-dimensional x-ray images (for example, x-ray data sets). Two- and three-dimensional x-ray data sets (for example, of the knee of the patient 1 who is on an operating table 9) are acquired by means of the x-ray device 2.

The three-dimensional x-ray data sets are acquired, in that first a plurality of two-dimensional x-ray images of a determined area of the patient 1 are recorded by the x-ray device 2. A plurality of two-dimensional x-ray images of the determined area (for example, knee) are thereby recorded at various angles by the C-arm of the x-ray device 2. The two-dimensional x-ray data sets acquired in this manner are projected in at least one three-dimensional area by a computer using known projection matrices. In this manner, a three-dimensional area with a determined volume can be recorded and displayed.

Furthermore, the inventive operation system comprises a surgical instrument 3 that is directed by a robot 4 or manipulator, and a position detection device 8.

The position detection device 8 is a component of the position detection system and serves to detect the positions of the surgical instrument 3 and the x-ray device 2. For this reason, a first position marker 5 is applied to the x-ray device 2, and a second position marker 6 is applied to the surgical instrument or, respectively, the robot 4 that directs the surgical instrument 3.

The inventive operation system additionally comprises a control device 12 to automatically control or, respectively, monitor the robot or, respectively, manipulator 4.

According to the present invention, the online imaging enables the direct imaging of the organs of interest in the operation environment, meaning that the necessary x-ray images are acquired during the operation; the acquisition of x-ray images prior to the operation (pre-operative), which is associated with an elaborate registrations procedure for implementing the operational procedure, is thus dispensed with.

The problem that subject movements, deformations, repositionings, and so on, of the area of concern can ensue in the time between the pre-operative imaging and the operational procedure or, respectively, during the operational procedure is compensated for, since the area of concern is correctly and therefore geometrically exactly imaged.

To establish the relationship between the spatial position of the surgical instrument 3 and the spatial position of the area of the patient whose x-ray image is two- or, respectively, three-dimensionally displayed, the relationship between the second position marker 6 (applied to the robot or, respectively, surgical instrument 3) and the operating area (for example, the point) of the surgical instrument 3 must be known.

Furthermore, the relationship between the first position marker 5 (applied to the x-ray device 2) and the spatial position of the acquired x-ray image must be known. This relationship is extensively specified in the patent application by Siemens AG, “Verfahren zur Ermittlung einer Koordinatentransformation für die Navigation eines Objektes”, with the application number DE 10042963.7.

Via these known relationships, the direct spatial relationship between the x-ray image (x-ray data set), the patient (or, respectively, the appropriate area or, respectively, organ), and the surgical instrument can be established and shown on a screen (display device 11), such that the instrument can be directly controlled and monitored by means of the image monitoring. These spatial relationships can also be processed as specified by the control device 12 to control and monitor the robot/manipulator 4.

In an advantageous embodiment of the present invention, a third position marker 7 is applied in the area of the patient 1 (for example, the knee) that is detected by the x-ray device. This position marker 7 is likewise registered by the position detection device. It is thereby achieved that possible movements of the patient during the procedure can be monitored in the graphical display of the surgical instrument 3, in that these movements are taken into account by the processing device 10 in the display of the instrument 3 in the display of the patient 1 and output on the screen 11. In this manner, it is ensured that the indicated current spatial relationship between the recorded area of the patient 1 and the surgical instrument 3 further coincides with the actual spatial relationship.

FIG. 2 shows a function diagram that shows the procedure of the control of the surgical instrument according to the present invention.

On the one hand, the x-ray device 2 provides a two- or, respectively, three-dimensional x-ray image 12. On the other hand, a spatial position 13 of the x-ray device 2 is determined by the position detection system. The actual spatial position of the area that is detected with the x-ray image or, respectively, the display of this x-ray image is determined from the spatial position 13 of the x-ray device (or, respectively, its position marker), for example as in the cited patent application “Verfahren zur Ermittlung einer Koordinatentransformation für die Navigation eines Objektes”.

Furthermore, a position 15 of the surgical instrument or, respectively, of the robot or manipulator that directs the surgical instrument is detected by the position detection system.

The acquired x-ray image 12, the position 14 of the recorded area, and the position 15 of the surgical instrument affect the control 16 or, respectively, the direction of the surgical instrument (position 15 of the surgical instrument), and thus affect the operational procedure 17. The control 16 of the surgical instrument can thereby ensue manually via the surgeon, manually by means of a manipulator, or automatically via a robot.

1. Operation system for implementation of operational procedures on a patient (1) by means of a surgical instrument (3), with:
an x-ray device (2) to record an x-ray image of at least one area of the patient (1), whereby the acquired x-ray image is graphically displayed by an output device (11),
a position detection system to detect the position of the x-ray device (2) during the recording, and to continually detect the position of the surgical instrument (3) during the operational procedure, and
a processing device (10) to process the detected positions and continually mix an image of the surgical instrument (3) into the displayed x-ray image, whereby the mixing of the x-ray image and the image of the surgical instrument ensues in correct positional arrangement according to the actual spatial positions between the position of the recorded area of the patient (1) and the position of the surgical instrument (3), and the surgical instrument (3) is directed by a robot or manipulator (4).

2. Operation system according to claim 1, characterized in that the position detection system comprises a first position marker (5) that is applied to the x-ray device (2), a second position marker (6) that is applied to the surgical instrument (3), and a position detection device (8) to detect the respective positions of the x-ray device (2) and the surgical instrument (3).

3. Operation system according to claim 1 or 2, characterized by a third position marker (7) to detect the position of the minimum one area of the patient (1) via the position detection device.

4. Operation system according to claim 1, 2, or 3, characterized by a control device to automatically control the robot (4) or, respectively, to monitor the manipulator.

5. Method for controlling a surgical instrument (3) during an operational procedure on a patient (1), with the steps:
acquisition of an x-ray image of at least one area of the patient (1), whereby the recorded x-ray image is graphically displayed,
detection of the position of the x-ray device (2) during the recording, and continual detection of the position of the surgical instrument (3) during the operational procedure,
processing of the detection positions and continual mixing of an image of the surgical instrument (3) into the displayed x-ray image, whereby the mixing of the x-ray image and the image of the surgical instrument ensues in correct positional arrangement according to the actual spatial positions between the position of the recorded area of the patient (1) and the position of the surgical instrument (3), and
direction of the surgical instrument (3) by a robot or manipulator (4).

6. Method according to claim 5, characterized by the detection of the position of the minimum one area of the patient (1) via the position detection device (8).

7. Method according to claim 5 or 6, characterized by an automatic control of the robot (4) or, respectively, monitoring of the manipulator (4). recorded area of the patient (1) and the position of the surgical instrument (3),
characterized in that the determination of the spatial position of the area of the patient detected with the x-ray image (12) ensues from a relationship between the x-ray device (2) and the information comprised within the x-ray image (12) and that spatial position of the x-ray device (2) detected during the acquisition by means of the position detection system (8).

7. Method according to claim 6, characterized by the direction of the surgical instrument (3) by a robot or manipulator (4), using the image of the surgical instrument (3) mixed into the displayed x-ray image.

8. Method according to claim 6 or 7, characterized by the detection of the position of the minimum one area of the patient (1) via the position detection device (8).

9. Method according to claim 6, 7, or 8, characterized by an automatic control of the robot (4) for automatic implementation of determined procedures or, respectively, automatic monitoring of a procedure area.
